(19) United States
(12) Patent Application Publication
(10) Pub. No.: US 2009/0204080 A1
(43) Pub. Date: Aug. 13, 2009

(54) TWO-WAY VALVE CONNECTOR

(75) Inventors: Patrick Balteau, Evelette (BE); Giampiero Pesci, Sondrio (IT)

Correspondence Address: BAXTER HEALTHCARE CORPORATION, 1 BAXTER PARKWAY, DF2-2E, DEERFIELD, IL, 60015, US

(73) Assignees: BAXTER INTERNATIONAL INC., DEERFIELD, IL (US); BAXTER HEALTHCARE S.A., ZURICH (CH)

(21) Appl. No.: 12/029,869

(22) Filed: Feb. 12, 2008

(51) Int. Cl. A61M 39/22

(52) U.S. Cl. 604/249; 251/149.1

(57) ABSTRACT

A medical fluid valve is provided that includes a housing having an inlet and outlet, a septum located at the inlet, and an arm connected to the housing. The arm can also include a plurality of teeth. When the housing is in a non-accessed position, the plurality of teeth can be positioned to prevent inadvertent access to a container. Furthermore, when the housing is in an accessed position, the plurality of teeth can prevent disconnection.

### BACKGROUND

Luer activated devices (“LAD”) or valves (“LAV”) are commonly used in association with medical fluid containers and medical fluid flow systems that are connected to patients or other subjects undergoing diagnostic, therapeutic or other medical procedures. A LAD can be attached to or part of a fluid container or a medical fluid flow system to simplify the addition of fluids to or withdrawal of fluids from the fluid flow system.

Within the medical field there are a wide variety of medical fluid flow systems, serving a variety of functions. One of the more common uses of LADs or LAVs is in association with fluid flow systems that are used for the intravenous administration of fluids, such as saline, antibiotics, or other medically-related fluids, to a patient. These flow systems are commonly referred to as intravenous or “IV” fluid administration sets, and use plastic tubing to connect a phlebotomized subject to one or more medical fluid sources, such as intravenous solution or medicament containers.

Typically, such intravenous administration sets include one or more LADs or LAVs providing needless access to the fluid flow path to allow fluid to be added to or withdrawn from the IV tubing. The absence of a needle for injecting or withdrawing fluid has the important advantage of reducing the incidence of needle stick injuries to medical personnel. A LAD or LAV typically includes a tapered female luer component, such as the inlet into a valve housing, which accepts and mates with a tapered male luer of a medical infusion or aspiration device, such as a needleless syringe or an administration set tubing brand.

There are certain characteristics and qualities of LADs or LAVs that are desirable. For example, the LAD or LAV should provide a sufficient microbial barrier for the full service life of the valve. It is desirable that the microbial barrier be conducive to the application of standard aseptic techniques preformed by clinicians during the use of the device. For example, the geometry of the LAD or LAV should be such that it is easily swabbable and reduces the potential of entrapping particulates or contaminants that cannot be cleanly swabbed clear prior to use.

In most situations it is preferred that the LAD or LAV be dimensioned to be completely activated by a wide range of ISO compliant male luer lock adaptors. Another desirable characteristic of a LAD or LAV is the ability to seal against pressure contained within a fluid system to which the LAD or LAV is connected. Yet another desirable characteristic of a LAD or LAV is the sterilization of the LAD or LAV before usage.

However, even after meeting these desirable characteristics, many LAD or LAV designs are still prone to misuse or inadvertent use and possible resulting contamination. For example, medical personnel operating a LAD or LAV may inadvertently access a container or port on a container before the container or port is properly sterilized. Moreover, medical personnel may inadvertently disconnect a LAD or LAV that has already accessed a container or port on a container. Further, many LAD-port connections may be prone to contamination before connection and during connection due to limited full service life of the LAD or port.

A need accordingly exists for a medical valve or device that can prevent access to a port or container and prevent disconnection after access to the port or container. A need also exists for a more reliable, user-friendly medical fluid valve or device. Moreover, a need exists for a medical fluid assembly that can decrease risk of contamination be during the service life of the assembly.

As described more fully below, the fluid access devices of the present disclosure provide advances in the safe, user-friendly and efficient administration or withdrawal of medical fluids to or from a container or fluid flow system.

### SUMMARY

The present disclosure generally provides a LAD that accesses a port on a fluid container to allow a male luer to inject and remove fluids to or from the container through the LAD. The valve has a lock that prevents the valve from inadvertently accessing the port. The same lock also prevents removal of the valve once the port is accessed. The LAD can also be constructed and packaged already locked to the port to keep the port and container sterile before the LAD is luer activated. Specific embodiments of the present disclosure are provided as follows.

In an embodiment of the present disclosure, a medical fluid valve for a container is provided. The medical fluid valve includes a housing. The housing includes an inlet and an outlet. The medical fluid valve also includes a luer accessible valve located at the inlet. Additionally, the valve includes an arm connected to the housing. The arm can also include a plurality of teeth. When the housing is in a non-accessed position, the plurality of teeth are positioned to prevent inadvertent access to a container. When the housing is in an accessed position, the plurality of teeth prevent disconnection.

In another embodiment of the present disclosure, a medical fluid assembly is provided. The medical fluid assembly includes a container that has a port connected to it. The fluid assembly also includes a valve. The valve includes a housing that has an arm connected to it. The arm includes a first tooth that is configured to hold the housing in a non-accessed position with respect to the container. The arm also includes a second tooth that is figured to hold the housing at an accessed position with respect to the container.

In a further embodiment of the present disclosure, a method for accessing a fluid container is provided. The method includes locking a valve in a non-accessed position with respect to the fluid container. The method also includes translating the valve from the non-accessed position to an accessed position. The method further includes locking to valve in the accessed position with respect to the fluid container.

It is an advantage of the present disclosure to provide a medical valve that can prevent inadvertent access to a fluid container.

It is another advantage of the present disclosure to provide a medical valve that can prevent disconnection after the valve accesses the fluid container.

It is a further advantage of the present disclosure to provide an improved locking mechanism for a medical valve.

It is another advantage of the present disclosure to provide a medical valve that can be adapted to connect to different connectors.

It is another advantage of the present disclosure to provide a medical valve that can maintain a long term, non-activated connection to a fluid container without fluid contact.

It is another advantage of the present disclosure to provide a visual indication of activation between a medical valve and a fluid container.

It is yet another advantage of the present disclosure to provide a medical fluid assembly that is user-friendly.

It is still a further advantage of the present disclosure to provide a medical fluid assembly configured to decrease the risk of contamination.

It is another advantage of the present disclosure to provide a medical fluid assembly that can withstand different methods of sterilization

It is another advantage of the present disclosure to provide a medical fluid assembly that can maintain a sterile barrier during transport.

Moreover, it is an advantage of the present disclosure to provide an improved method for accessing a container for the injection and withdrawal of a liquid to and from the container.

Still another advantage of the present disclosure is to provide a method for accessing a container that maintains a sterile environment before and after access to the container.

Additional features and advantages are described herein, and will be apparent from, the following Detailed Description and the figures.

### BRIEF DESCRIPTION OF THE FIGURES

FIG. 1 is a perspective view of one embodiment of a medical fluid valve of the present disclosure.

FIG. 2 is a side elevation view of one embodiment of a medical fluid container including a medical fluid assembly of the present disclosure.

FIG. 3 is a partial side elevational view of one embodiment of a medical fluid container of the present disclosure including a fluid assembly and a removable film surrounding the fluid assembly.

FIG. 4 is a partial side elevation view of one embodiment of a medical fluid container of the present disclosure including a medical fluid assembly in a non-accessed arrangement with one port partially sectioned and one port not sectioned.

FIG. 5 is a partial side elevation view of one embodiment of a medical fluid container of the present disclosure including a medical fluid assembly in a non-accessed arrangement with one port partially sectioned and one port not sectioned.

FIG. 6 is a perspective view of one embodiment of a medical fluid valve of the present disclosure including a film or tape.

FIG. 7 is a perspective view of one embodiment of a cap portion of a medical fluid valve of the present disclosure.

FIG. 8 is a perspective view of one embodiment of a septum portion of a medical fluid valve of the present disclosure.

### DETAILED DESCRIPTION

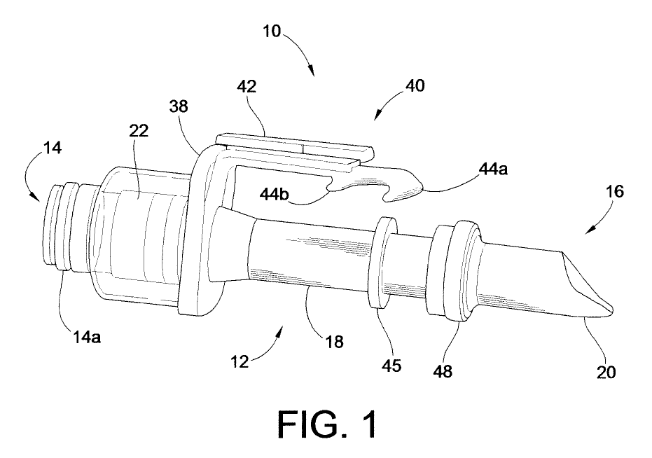

Referring now to the drawings, FIG. 1 illustrates one embodiment of a luer-activated device (“LAD”) or valve 10 of the present disclosure. The valve 10 is made of a rigid material, such as rigid plastic or other suitable material. The plastic materials can withstand one or more sterilization method such as, for example, gamma radiation, ethylene oxide, and steam sterilization.

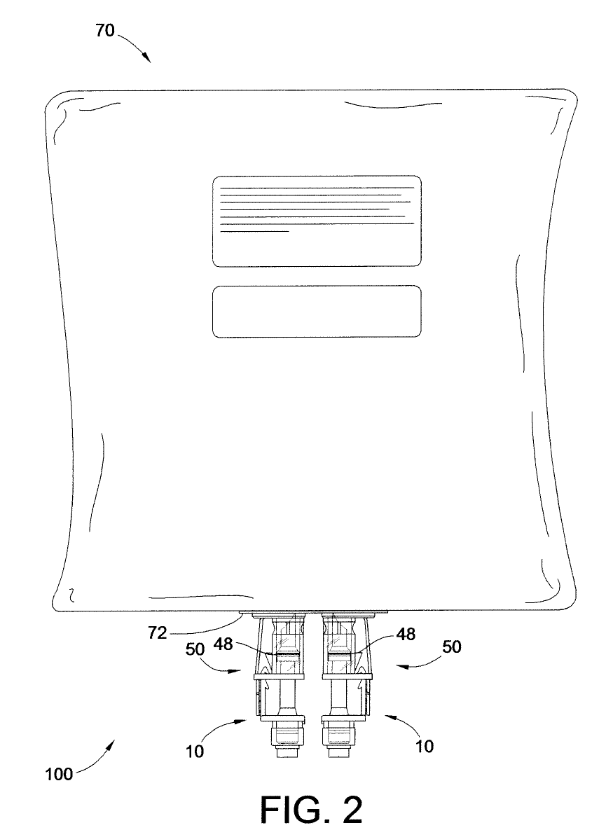

Valve 10 includes a valve housing 12, an inlet 14, an outlet 16 and a tube 18 separating inlet 14 from outlet 16. The terms “inlet” and “outlet” are for purposes of description only and are not to be interpreted as limiting the valve 10 to applications involving fluid flow in a particular direction, e.g., from the inlet 14 to the outlet 16, because valve 10 according to the present disclosure may be used in applications involving fluid flow from the inlet 14 to the outlet 16 or from the outlet 16 to the inlet 14. Valve 10 may also be formed as an integral part of a larger structure without departing from the present disclosure, as will be discussed below with reference to FIG. 2.

Outlet 16 is configurable to connect to multiple types of fluid flow systems or containers, the exact configuration of outlet 16 varying according to the type of fluid flow system to which it is to be connected. In the embodiments illustrated herein, outlet 16 has a spike tip 20 configured to puncture a solution barrier and establish fluid communication with the interior of a container. Depending on the type of fluid flow system or container connected to valve 10, spike tip 20 may be configured, for example, to penetrate a septum, rupture a membrane, or compress a hollow plug to connect to and access a fluid flow system or container to facilitate the injection or withdrawal of fluid from the fluid flow system or container.

Inlet 14 is configured to receive a generally mating connector (not pictured), such as male connector and in an embodiment a standard male luer. In various embodiments, inlet 14 and the male luer conform to ISO and/or ANSI standards. The male luer is generally tubular and has a substantially smooth outer surface that may be slightly tapered. The illustrated inlet 14 includes external threads 14a, so that a removable threaded locking connection may be established with a mating male luer having an annular collar or skirt member including internal threads. Other attachment mechanisms, such as a frictional engagement with a tapered male luer slip-fit can also be incorporated into the present LAD.

To control flow through housing 12, a hollow valve element or septum 22 is provided, which is generally configured to receive the male luer or other male connector. Septum 22 is molded as a unitary piece, typically from a deformable elastomeric material, such as silicone, such as ELASTOSIL silicone manufactured by Wacker of Adrian Mich., rubber, or a thermoplastic, such as Santoprene® thermoplastic, manufactured by Advanced Elastomer Systems, LP of Akron, Ohio.

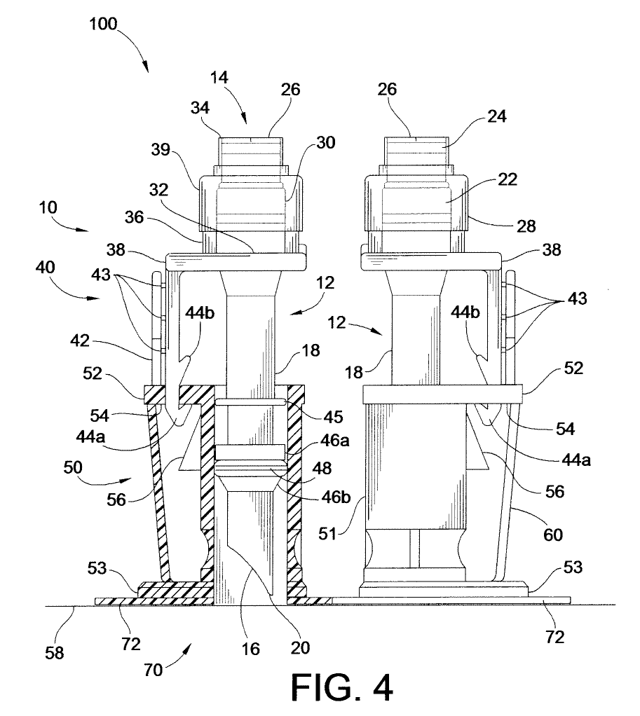

As seen in FIGS. 1 and 4, housing 12 includes a tube 18. Tube 18 is positioned to provide a fluid path between inlet 14 and outlet 16. Tube 18 is normally sealed or isolated from fluid flow when the septum tip 24 is in a closed condition. Septum 22 also provides a microbial barrier between the fluid path within tube 18 and atmosphere.

Housing 12 also includes or is attached to an arm 40 which is attached to a tab 42 and teeth 44a and 44b. Arm 40 is formed, e.g. molded, at least substantially parallel to tube 18 housing 12 and is connected to housing via a flange 38 extending radically from housing 12. Tab 42 is provided on the outside of arm 40 with respect to housing 12, while teeth 44a and 44b are formed on the inside of arm 40 with respect to housing 12. Tab 42 in one embodiment is connected to arm 40 by a frangible connection via thin frangible snaps 43 that snap off when the user twists or manipulates tab 42.

The valve housing 12 may further include at least one groove (not shown) formed on the exterior of housing 12, as a space between groove flanges 46a and 46b, to accept and seat an O-ring 48. O-ring 48 has a diameter greater than that of tube 18, which allows the O-ring 48 to form a sliding fluid tight seal with an interior wall of a container, or port on the container, to prevent fluid leaks when valve 10 is connected to a port or container. O-ring 48 can be made from a deformable elastomeric material, such as neoprene, rubber, silicone or a thermoplastic elastomer.

Referring now to FIG. 4, medical fluid valve 10 is shown as a part of a medical fluid assembly 100, which also includes a port 50, made of a partially transparent or opaque rigid material, integrally formed on a bottom base 72 of container 70. Because these are integrally formed, bottom base 72 and port 50 are made of the same material. FIG. 2 illustrates valve 10 in a first position, in which a portion of valve 10 is located within port 50 but has not yet accessed container 70. In this first position, gasket 48 provides a fluid tight seal that maintains a sterile environment within a sealed area between the gasket 48 and container 70. To assist in maintaining this sterile first position, medical fluid assembly 100 may be manufactured, sterilized and packaged with the valve 10 in the first position illustrated in FIG. 2.

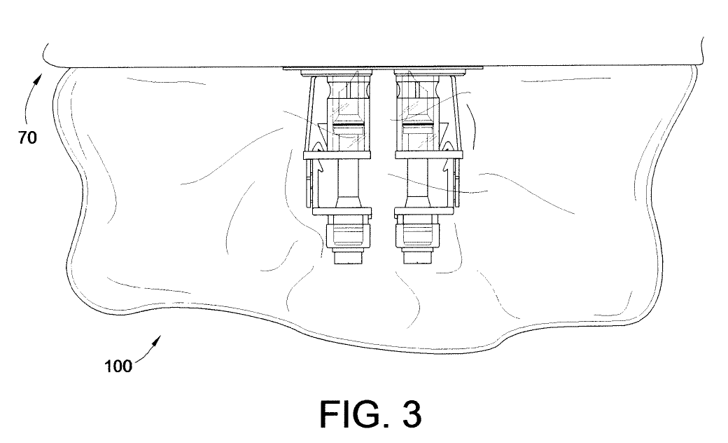

Referring to FIG. 3, fluid assembly 100 can be encased by a wrapping film which in an embodiment, also encases the container 70. In an embodiment the wrapping is vacuum wrapped around the assembly 100 and engages the assembly such that the wrapping holds the fluid assembly 100 in place or a non-accessed arrangement during storage and handling. In addition to being vacuum-wrapped, the fluid assembly may also be subjected to steam, super-heated water, dry heat, ethylene oxide (EtO), or Gamma sterilization methods to help prevent contamination. Such sterilization may occur before or after wrapping.

Referring to FIG. 4, the port 50 includes a port housing 51, a port flange 52 and a port arm 60. Port 50, like valve 10, is made of a rigid material, such as rigid plastic or other suitable material such as Polypropylene (PP), high-density polypropylene (HDPE), polysulfone, Poly(Styrene-Ethylene-Butadiene-Styrene) Elastomer (SEBS), Poly(Styrene-Butadiene-Styrene) (SBS), a blend of PP and SEBS, HDPE and SEBS, PP and SBS, HDPE and SBS, or any suitable functional material known to withstand heat sterilization methods (e.g. steam, super-heated water, dry heat); or polyethylene (PE), polyvinyl chloride (PVC), acrylonitrile butadiene styrene (ABS), SEBS, SBS or any suitable functional material known to the subject matter expert for “cold” sterilization (e.g. (EtO) or Gamma sterilzation). Port 50 can also be made of a partially transparent rigid material, as illustrated in FIGS. 2 and 3. Port flange 52 is formed, e.g. molded, at the distal end of port 50, opposite container 70. Port flange 52 includes a port aperture 54 configured and sized such that the arm 40, and teeth 44a and 44b, of valve 10 can pass through the port aperture 54 snugly.

Port arm 60 is formed or provided between port flange 52 and container 70 and connects the port flange 52 to the container 70 or to a base 53 of port 50 sealed to container 70. Port arm 60 supports port flange 52, such that a downward pressure applied to valve 10 will not manipulate or deform port flange 52. Arm 60 helps flange 52 to provide a physical resistance against which the user can move valve 10 relative to port 50. Port arm 60 may run substantially parallel to the port housing 51 or have a slight angle as illustrated in FIG. 4.

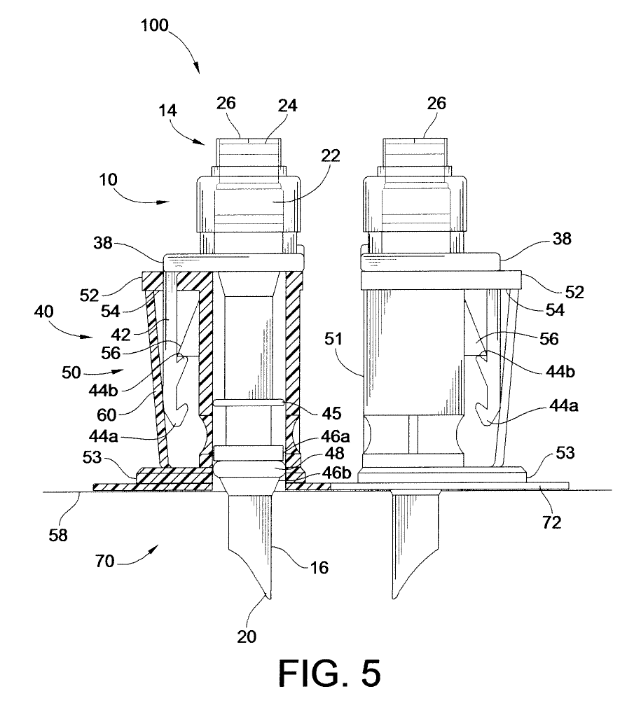

Port arm 60 may also be configured to provide support and/or limit the movement of the valve arm 40 when valve arm 40 reaches a second access position (FIG. 5), so that arm 40 will abut port arm 60 and prevent valve arm 40 from advancing further towards container 70. Port arm 60 tapers inward from the port flange 52 in the illustrated embodiment to provide such abutment.

A membrane 58 is bonded directly to bottom base 72 of container 70. Membrane 58 therefore seals the fluid contents within the container 70 until ruptured. Spike 20 ruptures membrane 58 to establish fluid communication between valve 10 and container 70. The membrane 58 serves as a one-time use membrane such that once ruptured, the membrane 58 cannot be resealed. As opposed to membrane 58, bottom base 72 does not extend into the interior of the port housing 51. Therefore, spike 20 only ruptures membrane 58, not base 72.

Alternatively, port 50 could be sealed in between inner and outer membrane layers of which container 70 is made provided port 50 is made from the same material as that of inner membrane layer of container 70.

Port 50 also includes a port tooth 56 in the illustrated embodiment. Port tooth 56 is formed on the flange 52 side of housing 51, such that port tooth 56 forms a snap-lock fit with tooth 44b of valve arm 40. Port tooth 56 is angled as shown to provide the snap-lock fit to occur. For example, the port tooth 56 can have the illustrated saw tooth shape with short side positioned away from the port flange 52. The saw tooth shape of port tooth 56 is opposite to the barbed configuration of valve tooth 44a, is textured to meet the taper of port tooth 56 until the two teeth are moved to snap together. In operation, the opposing shapes of teeth 44a and 44b versus tooth 56 promote a desired snap-lock fit illustrated in FIG. 3 when valve 10 reaches the second, accessed, position.

As illustrated in FIG. 2, container 70 includes two ports 50 and two valves 10. The two ports allow recirculation after priming of the HD blood lines to ensure that all the air trapped in the blood lines is actually kept in the container. The two ports also allow redundant access to the container for reasons such as safety, or to allow a medication injection with a needleless syringe or the fitment of any compatible drug injection device (typically fitted with a male luer-lock fitment) when the other port is in use. Container 70 may have a hanger hole for a hook to hang the container 70 in an elevated position at or near a patient. When the container 70 is positioned at or near the patient, gravity forces the liquid inside the container 70 through the port 50 and valve 10 with an administration or disposable set used for dialysis, drug delivery, dialysis, nutritional delivery, or other solution infusion application. Container 70 may be constructed of a flexible material, such as PVC or a non-PVC plastic.

In the non-accessed position of FIG. 4, spike 20 rests inside port 50, with gasket 48 positioned inside port 50 to seal to port 50 and maintain a sterile environment between the seal point and container 70. Spike 20 as shown does not penetrate membrane 58 of port 50 in the first, non-accessed, position because of the position of valve arm 40 in relation to port flange 52 and container 70. More specifically, in the first position, tab 42 abuts port flange 52, preventing valve 10 from advancing into the port 50 and accessing fluid in the container 70. Tab 42 accordingly prevents inadvertent accessing of the container 70.

In the first position, a first tooth 44a on arm 40 has passed through port aperture 54 and rests in a locked position against flange 52. That is, once barbed tooth 44a passes through aperture 54, it cannot be pulled back out, locking valve 10 in the non-accessed position and preventing valve 10 from being pulled away from container 70. Tab 42 and tooth 44a of arm 40 accordingly lock valve 10 to port 50, and the only direction valve 10 can move, when tab 42 is removed, is to puncture container 70.

To allow the valve 10 to advance from the first position, tab 42, of valve 10 is removed from arm 40, thereby unlocking valve 10 for advancement through port 50. As valve 10 is pressed through port 50 from the first position of FIG. 2 to the second, accessed, position illustrated in FIG. 5, spike tip 20 pierces port membrane 58 to provide valve 10 access to container 70.

Valve 10 is also locked in place with respect to the container 70 in the second position. As seen in FIG. 5, to lock valve 10 in the second position, arm 40 advances through port aperture 54 and along port housing 51 until second tooth 44b passes through aperture 54 and snap-fits to port tooth 56. At the second position, the snap-fit between second tooth 44b and port tooth 56 prevents disconnection of valve 10 from container 70 after valve 10 has accessed container 70. Moreover, gasket 48 remains in port 50 and therefore maintains the sterile seal that existed when valve 10 was in the non-accessed position.

In the illustrated embodiment, the length of arm 40 prevents valve 10 from advancing further into container 70. As illustrated in FIG. 2, arm 40 is sized such that, in the first non-accessed position, the arm 40 extends so as to reach port 50 on container 70 and terminates prior to spike tip 20 accessing container 70. FIG. 5 illustrates that arm 40 is sufficiently short such that when second tooth 44b snap fits to port tooth 56, the housing flange 38 abuts port flange 52, making further advancement of valve 10 impossible. If arm 40 is longer than that illustrated in FIG. 3, housing flange 38 and port flange 52 may not abut before arm 40 contacts or disturbs container 70 or base 53. As mentioned, port arm 60 can be tapered such that arm 40 is engaged frictionally by port arm 60 before contacting container 70. The port arm 60 configuration also guides the arm 40 to extend closely along the port tube so that the teeth 44b and 56 are certain to engage. By preventing any further advancement of arm 40, valve 10 is rendered incapable of either advancing or retracting (disconnecting) after reaching the second position (via housing flange 38 to port flange 52 and tooth 44b to tooth 56 contact).

To inject or withdraw fluid through valve 10 in the second, accessed, position, a male luer or connector (not pictured) is introduced to septum 22. When the male luer or connector pierces slit 26 of septum 22, septum tip 24 folds or deforms to open the first aperture 26 within inlet 14, allowing the male luer or connector to withdraw fluid from container 70 or allowing fluid to flow through the male luer, through valve 10, and into container 70.

In the illustrated embodiment, valve 10 includes a tube flange 45 extending radially from tube 18 near gasket 48. Tube flange 45 functions to maintain co-axiality of valve 10 and more specifically tube 18 of said valve 10 into the cylindrical portions of access port 50 sealed on the outside surface of membrane 58. This will prevent the valve from being moved laterally and therefore breach the leak proof configuration of the “spike 18 to gasket 48 assembly” when press fitted into the cylindrical portions of said port 50. The design of valve 10 and access port 50 ensures the fitment to be leak proof (as tested by under water pressurization of the assembled valve and port) and bacterial-proof (as tested by immersion ingress testing of the bag-port-valve unit).

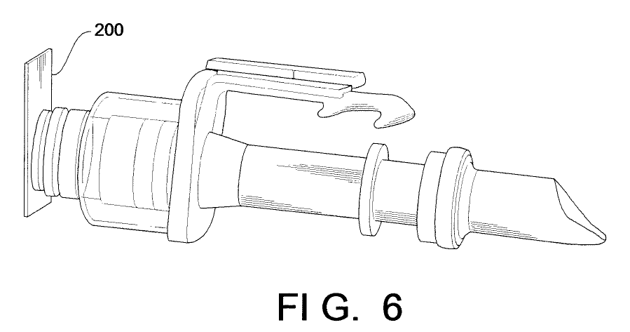

In an alternative embodiment shown in FIG. 6, a removable film or tape 200 is placed over the inlet 14 to prevent contamination of the septum tip 24 and inlet 14.

Various lad connectors could be substituted for the slit septum. Examples include ICU Medical's CLAVE® valve shown in U.S. Pat. No. 5,685,866; Alaris Medical Systems' SMARTSITE® valve shown in U.S. Pat. No. 6,290,206; and Nypro's Luer-Activated valves shown in U.S. Pat. No. 5,775,671.

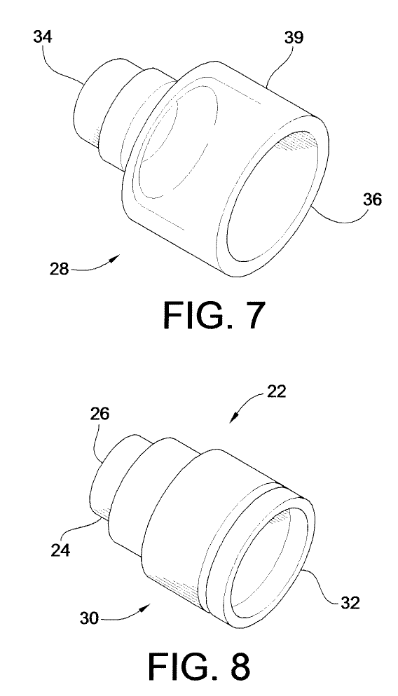

In an alternative embodiment illustrated in FIGS. 7 and 8, septum 22 is mounted inside a cap 28 of housing 12 of valve 10. Cap 28 is open at both ends 34 and 36. Cap 28 has a wall 38 that is sized, e.g., for press-fit with septum 22. Or, wall 39 is conical or tapered, such that the wall diameter of the cap 28 increases from the cap inlet 34 to the cap outlet 36 to match the diameter of at least a portion of septum 22, fixing septum 22 to within cap 28.

An upper end or tip 24 of septum 22 defines a normally closed resealable first aperture or slit 26. Septum 22 is fixed within the valve 10, such that septum tip 24 creates a seal at inlet 14 of valve 10 about the male luer when the luer is inserted into inlet 14. Portion 30 having a larger radius than tip 24 extends from the tip 24 to an end 32 of the septum 22. End 32 may define an open-ended portion, a slit or a hole. Portion 30 is tubular as shown and sized to fit snugly within inlet 14, so that when septum 22 receives a male luer or connector, the male luer or connector cannot translate the entire septum 22 into the tube 18. Instead, end 32 will remain substantially static while the first aperture 26 opens and the septum tip 24 deforms to allow the male luer to withdraw or inject fluid through the hollow septum 22.

In another embodiment, septum tip 24 has a substantially flat or slightly outwardly curved outside surface that can be easily wiped with antiseptic, which aids in preventing contamination during use.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. A medical fluid valve for a container comprising:
a housing including an inlet and an outlet;
a luer accessible valve positioned at the inlet; and
an arm connected to the housing, the arm including a plurality of teeth, the teeth positioned on the arm so as to (i) prevent inadvertent access to the container when the housing is positioned in a non-accessed position with respect to the container and (ii) prevent disconnection after the housing is positioned in an accessed position with respect to the container.

2. The medical fluid valve of claim 1, the valve is configured to receive a male luer.

3. The medical fluid valve of claim 1, the housing including an outlet configured to access a container.

4. The medical fluid valve of claim 1, which includes a gasket positioned on the housing, the gasket sized to seal with a port located on the container.

5. The medical fluid valve of claim 1, the housing including a flange and an arm extending from the flange.

6. The medical fluid valve of claim 1, wherein at least one of the teeth is barbed to prevent the housing from being pulled away from the container after the tooth is forced through an aperture of a port fixed to the container.

7. The medical fluid valve of claim 1, the teeth including a first tooth configured to at least partially hold the housing at the non-accessed position and a second tooth configured to hold at least partially the housing at the accessed position.

8. The medical fluid valve of claim 1, the teeth including a first tooth configured to operate with a frangible tab connected to the arm to hold the housing at the non-accessed position and a second tooth configured to operate with a flange connected to the area to hold the housing at the accessed position.

9. The medical fluid valve of claim 1, the housing including a tube separating the inlet and the outlet, the arm extending generally parallel to the tube.

10. The medical fluid valve of claim 1, the inlet including at least one of: a removable film covering the inlet; and a threaded cap.

11. The medical fluid valve of claim 1, which includes a frangible tab removable to allow the housing to be moved from the non-accessed position to the accessed position.

12. The medical fluid valve of claim 1, the arm extending so as to reach a port located on the container but terminating prior to the outlet accessing the container.

13. The medical fluid valve of claim 1, the teeth including a tooth positioned to mate with an opposing tooth of a port located on the container to hold the housing at the accessed position.

14. A medical fluid assembly comprising:
a container;
a port connected to the container; and
a valve including a housing, an arm connected to the housing, the arm including a first tooth configured to at least partially hold the housing in a non-accessed position with respect to the container and a second tooth configured to at least partially hold the housing at an accessed position with respect to the container.

15. The medical fluid assembly of claim 14, the first tooth configured to operate with a frangible tab connected to the arm to hold the housing at the non-accessed position and the second tooth configured to operate with a flange connected to the area to hold the housing at the accessed position.

16. The medical fluid assembly of claim 14, the valve including a first flange, the port including a second flange, the first and second flanges positioned to mate when the housing is located at the accessed position.

17. The medical fluid assembly of claim 14, the port including a flange extending from the port, the flange defining an aperture, the aperture positioned to receive the arm.

18. The medical fluid assembly of claim 17, the port including a port arm extending between the flange and the container to support the flange.

19. A method for accessing a fluid container, the method comprising:
locking a valve in a non-accessed position with respect to the fluid container such that the valve cannot be pulled away from the container;
translating the valve from the non-accessed position to an accessed position; and
locking the valve in the accessed position with respect to the fluid container such that the valve cannot be removed from the container or further translated in the accessed direction.

20. The method of claim 19, which includes maintaining a seal between the valve and the container in non-accessed and accessed positions.

21. The method of claim 20, which includes maintaining a sterile environment between the valve and the container in non-accessed and accessed positions.

22. The method of claim 19, which includes removing a frangible tab from the valve before translating the valve from the non-accessed position to an accessed position.

23. The method of claim 19, which includes inserting a male luer into the valve and withdrawing or injecting fluid.
